(19) United States
(12) Patent Application Publication
(10) Pub. No.: US 2007/0038048 A1
(43) Pub. Date: Feb. 15, 2007

(54) COMBINATION SENSOR FOR DETERMINING PHYSIOLOGICAL PARAMETERS ON THE SKIN OF A PATIENT

(75) Inventor: Henning GERDER, Lübeck (DE)

Correspondence Address: MCGLEW & TUTTLE, PC, P.O. BOX 9227, SCARBOROUGH STATION, SCARBOROUGH, NY, 10510-9227, US

(73) Assignee: DRAGER MEDICAL AG & CO. KG, Lubeck (DE)

(21) Appl. No.: 11/458,136

(22) Filed: Jul. 18, 2006

(30) Foreign Application Priority Data
Aug. 12, 2005 (DE) 10 2005 038 147.2

(51) Int. Cl. A61B 5/00

(52) U.S. Cl. 600/323; 600/345

(57) ABSTRACT

A combination sensor for the simultaneous use of different sensors for determining physiological parameters on the skin of a patient. The combination sensor includes a sensor carrier (1) which can be mounted on the body of the patient. Sensors are integrated in the sensor carrier for determining different physiological parameters of the patient through the skin of the patient. A measured signal processing unit (9) is provided for the sensors integrated in the sensor carrier (1). A communications unit (6) is integrated in the sensor carrier (1) for the wireless transmission of the processed sensor measured data. An electrical energy source (7) is integrated in the sensor carrier (1) for the energy supply of the components arranged in the sensor carrier (1). This combination makes possible a largely undisturbed accessibility to the patient in routine clinical practice.

### CROSS REFERENCE TO RELATED APPLICATIONS

This application claims the benefit of priority under 35 U.S.C. § 119 of German Patent Application DE 10 2005 038 147.2 filed Aug. 12, 2005, the entire contents of which are incorporated herein by reference.

### FIELD OF THE INVENTION

The present invention pertains to a combination sensor for determining physiological parameters on the skin of a patient.

### BACKGROUND OF THE INVENTION

DE 10 2004 032 812 A1 discloses a combination sensor with a heat flux sensor and an acoustic sensor.

It is known to mount various sensors separately for measuring physiological parameters on the skin of a patient, e.g., ECG electrodes with adhesive surfaces, temperature sensors with fastening means or specially designed optoelectronic sending and receiving units on the finger for the noninvasive, photometric determination of the oxygen or carbon dioxide partial pressure in the blood of the patient. Presently, the individual measuring points and measuring sensors in practice are connected to a plurality of parallel feed lines separately from one another at different evaluating devices. The separate mounting of the sensors and especially the number of different feed lines often make free access to the patient more difficult. This is especially disturbing in the case of very small patients in routine clinical practice.

### SUMMARY OF THE INVENTION

Accordingly, the object of the present invention is to provide a device that makes it possible to use different sensors on the skin of a patient simultaneously and thus largely guarantees undisturbed accessibility to the patient.

According to the invention, a combination sensor is provided for determining physiological parameters on the skin of a patient. The combination sensor comprises a sensor carrier which can be mounted on the body of the patient. Sensors are integrated in the sensor carrier for determining different physiological parameters of the patient through the patient's skin. A measured signal processing unit is provided for the sensors integrated in the sensor carrier. A communications unit is integrated in the sensor carrier for the wireless transmission of the processed sensor measured data. An electrical energy source is integrated in the sensor carrier for the energy supply of the components arranged in the sensor carrier.

An essential advantage of the combination sensor appears in that cables do not lead to the individual sensors, such that, besides the considerably improved accessibility to the patient, measured signal disturbances due to artifacts, cable movements or contact disturbances due to tensile loads of the cable are eliminated.

The sensors may advantageously be arranged in the sensor carrier in such a way that, after mounting the combination sensor on the body of the patient, the sensors contact the skin of the patient directly.

At least one of the sensors may advantageously be an ECG (electrocardiogram) electrode.

At least one of the sensors may advantageously be a temperature sensor or an optoelectronic sending and receiving unit.

A patch plug may advantageously be provided for external, wired calibration and/or data balance by means of an externally connectable cable connection.

The sensor carrier may advantageously be designed in the form of a hinged finger, wrist, arm, foot, toe or calf clip.

The sensor carrier may advantageously be designed in the form of a tape or strip with a fastener, especially in the form of a textile or woven cuff with a snap fastener, and especially with a hook, snap-button or Velcro fastener.

At least one of the sensors integrated in the sensor carrier may be a transcutaneous or transdermal sensor that contacts the skin of a patient such that the sensor does not penetrate the skin. The transcutaneous or transdermal sensor can advantageously be an electrical, chemical or biochemical sensor. The transcutaneous or transdermal sensor may also be an electrical sensor for measuring electrical resistivity or conductivity of the skin of a patient.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

The only FIGURE is a schematic view of an exemplary embodiment of a combination sensor with a sensor carrier in the form of a finger clip.

### DESCRIPTION OF THE PREFERRED EMBODIMENT

Referring to the drawings in particular, a sensor carrier 1 is designed in the form of a finger clip, which is made, e.g., of a plastic with the components integrated therein. Three ECG electrodes 2 designed as surface electrodes are integrated, e.g., in the sensor carrier 1 as are a temperature sensor 3 as well as an optoelectronic sending and receiving unit 4a, 4b for the photometric measurement of the oxygen and carbon dioxide partial pressure in the blood of the patient. Possible other sensors are provided such as transcutaneously measuring optical, electrical or electrochemical sensors, which are used for the determination of physiologically relevant substance concentrations or other relevant, noninvasively measurable patient parameters such as blood pressure. All sensors are arranged in the sensor carrier 1 in such a way that they contact the skin after mounting the combination sensor on the body of the patient and preferably on the patient's extremities.

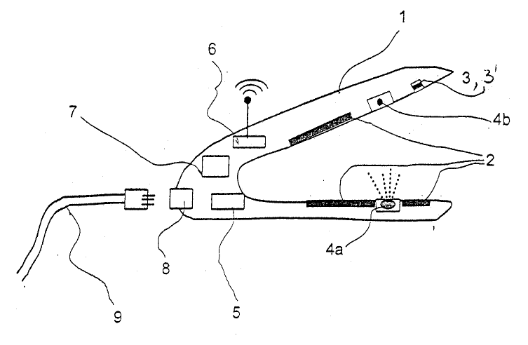

Referring to FIG. 1, according to an alternative embodiment at least one of the sensors integrated in the sensor carrier 1 may be a transcutaneous or transdermal sensor 3′ that contacts the skin of a patient such that the sensor does not penetrate the skin. The transcutaneous or transdermal sensor 3′ can advantageously be an electrical, chemical or biochemical sensor. The transcutaneous or transdermal sensor 3′ may also be an electrical sensor for measuring electrical resistivity or conductivity of the skin of a patient.

The electrical energy source 7, which is designed in the form of batteries, foil-type storage batteries or even as storage batteries equipped with an inductive charging switch, is used for supplying energy to the components arranged in the sensor carrier 1.

If necessary, the sensors are supplied by the energy source 7 and forward their measured signals to the measured signal processing unit 5, guided internally in the sensor carrier 1. Here, the measured signals are detected and processed, e.g., converted, taken into account and optionally filtered and corrected, according to well-known methods. The signals processed in this manner are finally sent by means of the communications unit 6 with antenna in a wireless manner to a receiver, e.g., at a nursing station in the hospital.

The data of the processed signals are then sent by means of radio transmission to various medical devices which have a corresponding receiving unit. The medical devices also include those with an integrated monitoring functionality, i.e., e.g., anesthesia workplaces, incubators, open patient care units and monitoring monitors. A combining and accounting can take place in the devices with measured data obtained in another way and device settings. It is also possible to send the data via radio indirectly via medical devices or directly by the combination sensor to a central monitoring station or to feed them into a wireless (WLAN) or wired (LAN) network.

The combination sensor is specially equipped with a patch plug 8 for an external cable connection 9 for a calibration of the combination sensor overall or by individual sensors and/or for data balance with optional data transmission via cable.

Reduction in the number of previously separate cable connections leading to the individual sensors with accompanying reduction in possible measured signal disturbances and formation of artifacts, the integrated measured signal processing for a plurality of sensors in one component, the joint data composite for a plurality of sensors, and the more efficient application immediately of a plurality of sensors with a single device should be mentioned as decisive advantages of the combination sensor.

While a specific embodiment of the invention has been shown and described in detail to illustrate the application of the principles of the invention, it will be understood that the invention may be embodied otherwise without departing from such principles.

What is claimed is:

1. A combination sensor for determining physiological parameters on skin of a patient, the combination sensor comprising:
a sensor carrier which can be mounted on the patient;
sensors integrated in said sensor carrier for determining different physiological parameters of the patient through the skin of the patient;
a measured signal processing unit for processing sensor signals of said sensors, said measured signal processing unit being integrated in said sensor carrier to provide processed sensor measured data;
a communications unit integrated in said sensor carrier for wireless transmission of the processed sensor measured data; and
an electrical energy source integrated in said sensor carrier for supplying energy to one or more of said sensors, said processing unit and said communications unit arranged in said sensor carrier.

2. A combination sensor in accordance with claim 1, wherein said sensors are arranged in said sensor carrier in such a way that, after mounting the combination sensor on the body of the patient, at least one of said sensors contact the skin of the patient directly.

3. A combination sensor in accordance with claim 1, wherein at least one of said sensors is an ECG (electrocardiogram) electrode.

4. A combination sensor in accordance with claim 1, wherein at least one of said sensors is a temperature sensor or an optoelectronic sending and receiving unit.

5. A combination sensor in accordance with claim 1, further comprising: externally connectable cable connection providing a patch plug for external, wired calibration and/or data balance.

6. A combination sensor in accordance with claim 1, wherein said sensor carrier is designed in the form of a hinged finger, wrist, arm, foot, toe or calf clip.

7. A combination sensor in accordance with claim 1, wherein said sensor carrier comprises a tape or strip with a fastener.

8. A combination sensor in accordance with claim 7, wherein said tape or strip comprises a textile or woven cuff and said fastener comprises a snap fastener.

9. A combination sensor in accordance with claim 7, wherein said fastener comprises a snap fastener, a hook fastener, snap-button or Velcro fastener.

10. A combination sensor in accordance with claim 1, wherein at least one of said sensors integrated in said sensor carrier is a transcutaneous or transdermal sensor that contacts the skin of a patient such that the sensor does not penetrate the skin.

11. A combination sensor in accordance with claim 10, wherein said transcutaneous or transdermal sensor is an electrical, chemical or biochemical sensor.

12. A combination sensor in accordance with claim 11, wherein said transcutaneous or transdermal sensor is an electrical sensor for measuring electrical resistivity or conductivity of the skin of a patient.

13. A sensor arrangement for determining a physiological parameter during connection to a body of a patient, the sensor arrangement comprising:
a sensor carrier;
a first sensor integrated in said sensor carrier for determining a physiological parameter of the patient;
a second sensor integrated in said sensor carrier for determining a physiological parameter of the patient;
a measured signal processing unit for processing sensor signals of said first sensor and said second sensor, said processing unit being integrated in said sensor carrier to provide processed sensor measured data;
a communications unit integrated in said sensor carrier; and
an electrical energy source integrated in said sensor carrier for supplying energy to one or more of said first sensor, said second sensor, said processing unit and said communications unit arranged in the sensor carrier, said sensor carrier being connectable with the body of the patient with each of said first sensor and second sensor in contact with or closely adjacent to skin of the patient.

14. A sensor arrangement according to claim 13, wherein said communications unit integrated in said sensor carrier includes wireless transmission means for the wireless transmission of the processed sensor measured data.

15. A combination sensor in accordance with claim 13, wherein said sensors are arranged in said sensor carrier in such a way that, after mounting the combination sensor on the body of the patient, at least one of said sensors contact the skin of the patient directly.

16. A combination sensor in accordance with claim 13, wherein at least one of said first sensor and said second sensor is an ECG (electrocardiogram) electrode.

17. A combination sensor in accordance with claim 13, wherein at least one of said first sensor and said second sensor is a temperature sensor or an optoelectronic sending and receiving unit.

18. A combination sensor in accordance with claim 13, further comprising: a wire or cable connection for external, wired calibration, data transfer and/or data balance.

19. A combination sensor in accordance with claim 13, wherein said sensor carrier is designed in the form of a hinged clip.

20. A combination sensor in accordance with claim 13, wherein said sensor carrier comprises a tape or strip with a fastener.

21. A combination sensor in accordance with claim 20, wherein said tape or strip comprises a textile or woven cuff and said fastener comprises a snap fastener.

22. A combination sensor in accordance with claim 20, wherein said fastener comprises a snap fastener, a hook fastener, snap-button or Velcro fastener.

23. A sensor arrangement for determining a physiological parameter during connection to a body of a patient, the sensor arrangement comprising:
a sensor carrier including means for mounting or connecting the carrier to the body of the patient;
a plurality of sensors integrated in said sensor carrier for respectively determining one or more physiological parameters of the patient;
a measured signal processing unit for processing sensor signals of said sensors, said processing unit being integrated in said sensor carrier to provide processed sensor measured data;
a communications unit integrated in said sensor carrier; and
an electrical energy source integrated in said sensor carrier for supplying energy to one or more of said sensors, said processing unit and said communications unit, said sensor carrier being connectable with the body of the patient via said means for mounting whereby at least one of said sensor is in operative connection with the skin of the patient.
